(19) United States
(12) Patent Application Publication
(10) Pub. No.: US 2007/0027424 A1
(43) Pub. Date: Feb. 1, 2007

(54) Tampon applicator for a feminine hygiene product providing proper vaginal placement

(75) Inventors: Diana Lynne Gann, Lebanon, OH (US); Thomas Ward Osborn III, Clifton, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/190,647

(22) Filed: Jul. 27, 2005

(51) Int. Cl. A61F 13/20

(52) U.S. Cl. 604/16

(57) ABSTRACT

A tampon applicator for placing a feminine hygiene tampon into a vagina which has a tampon, a tampon housing member, a cover member, and a tampon positioning member. The tampon is partially housed within the tampon housing member. The tampon extends beyond the tampon housing member. The cover member covers at least a portion of the tampon housing member. The tampon housing member is telescopically mounted within the cover member. The tampon positioning member positions the tampon within the tampon housing member. The tampon positioning member can be disposed inside the tampon housing member and can be co-axial with the tampon. The tampon positioning member can be fixedly joined to the cover member. Alternatively, the tampon positioning member can be fixedly joined to the tampon housing member.

### FIELD OF INVENTION

This invention relates to a tampon applicator, particularly, to a tampon applicator providing proper placement of a tampon within the vaginal cavity.

### BACKGROUND OF THE INVENTION

Tampon applicators have been used for inserting a tampon into a vaginal cavity to prevent leakage of menstrual fluids or other fluids. In the field of tampon applicators, “push” type tampon applicators are well known. These applicators generally comprise, in a simple form, a pair of coaxial hollow cylinders or tubes. The larger outer tube serves as a vaginal insertion tube and comprises a tampon. The smaller inner tube serves as a plunger which ejects the tampon from the outer tube.

It has been recognized that tampon applicators of the type described above have certain deficiencies which can be readily apparent to many users of such devices. In general, the position of the tampon within the vaginal cavity can directly impact the tampon's efficiency of reducing and/or eliminating leakage. For example, conventional “push” type applicators can place a tampon too high in the vaginal cavity. The higher the tampon is placed, the greater the chance of leakage due to the tampon being above the major path of fluid flow. In other words, menstrual fluids can pass by the tampon without being absorbed.

In addition, if the position of the tampon is too high in the vaginal cavity, the tampon can extend into portions of the vaginal cavity, where, for reasons of the size and mechanical properties of the tampon, the tampon can be negatively affected by the vaginal organs, further reducing the tampon coverage of menses flow in the vaginal cavity. Moreover, current tampon applicators are designed to “push” the tampon out of the outer tube so that the tip of the tampon is substantially higher than the outer tube. This often causes the tampon to be deflected by the cervix, resulting in an off-centered position of the tampon, and, thus, in possible leakage from the vaginal cavity.

Alternatively, a tampon can be placed too low in the vaginal cavity. When the tampon is placed too low in the vagina cavity, the too low position of the tampon can cause bodily discomfort to the wearer because of the pressure exerted from the sphincter muscles against the tampon. Also, if the tampon is placed too low, accidental expulsion of the tampon is a highly undesired risk.

To solve the above problem of positioning the tampon, it would be beneficial to provide a tampon applicator which properly places the tampon low in the vaginal cavity.

### SUMMARY OF THE INVENTION

The present invention encompasses a tampon applicator for positioning of a tampon. The tampon applicator comprises a tampon, a tampon housing member, a cover member, and a tampon positioning member. The tampon is partially housed within the tampon housing member. The tampon extends beyond the tampon housing member. The cover member covers at least a portion of the tampon housing member. The tampon housing member is telescopically mounted within the cover member. The tampon positioning member positions the tampon within the tampon housing member. The tampon positioning member is disposed inside the tampon housing member and is co-axial with the tampon. The tampon positioning member can be fixedly joined to the cover member. Alternatively, the tampon positioning member can be fixedly joined to the tampon housing member.

The present invention can also encompass a tampon applicator wherein the tampon housing member can be slideable over the tampon positioning member. In another embodiment, before expulsion of the tampon from the tampon applicator, the bottom end of the cover member can be positioned below the bottom end of the tampon housing member. In yet another embodiment, the tampon housing member can be joined to the cover member. Specifically, the exterior of the tampon housing member can be joined to the hollow interior of the cover member.

All documents cited are, in relevant part, incorporated herein by reference; the citation of any document is not to be construed as an admission that it is prior art with respect to the present invention.

### BRIEF DESCRIPTION OF THE DRAWINGS

FIG. 1a is a perspective view of a tampon applicator of the present invention.

FIG. 1b is an exploded view of the tampon applicator along the longitudinal axis illustrative of the present invention before the assembly of the tampon applicator.

FIG. 1c is a cross-sectional view of the tampon applicator of the present invention.

FIG. 2 is a perspective view of an alternative embodiment of the tampon applicator.

FIG. 3 is a perspective view of another alternative embodiment of the tampon applicator.

FIG. 4 is a perspective view of another alternative embodiment of the tampon applicator.

### DETAILED DESCRIPTION OF THE INVENTION

Section A will provide terms which will assist the reader in best understanding the features of the invention but not to introduce limitations in the terms inconsistent with the context in which they are used in this specification. These definitions are not intended to be limiting. Section B will discuss the tampon applicator of the present invention.

A. Terms

As used herein, the term “tampon” refers to any type of absorbent structure that can be inserted into the vaginal cavity or other body cavities for the absorption of fluid therefrom or for the delivery of active materials, such as medicaments or moisture. A tampon can be straight or non-linear in shape, such as curved along the longitudinal axis.

Generally, there are two types of tampons. The first type of tampon is a self-sustaining tampon. Tampons are generally “self-sustaining” in that they will tend to retain their general shape and size before use. A typical self-sustaining tampon is 35-60 mm long, the length measured from the top of the tampon to the base of the tampon along a longitudinal axis. The measurement to the base of the tampon does not include any overwrap, secondary absorbent member, or withdrawal cord which extends beyond the tampon's main absorbent material. A typical self-sustaining tampon is 5-20 mm wide corresponding to the largest cylindrical cross section. The width can vary along the length of the self-sustaining tampon.

The second type of tampon is an easily “deformable, fluid-permeable bag tampon”. The deformable, fluid-permeable bag tampon consists of absorbent chips, spheres, or fibers such that the fluid permeable bag tampon is readily deformable with a force of less than about 3 psi. The tampon is substantially deformable at pressures of less than about 1 psi; resulting in the tampon spreading or being easily indented when the pressure is applied from a surface of about 0.15 mm diameter.

As used herein, the terms “vaginal cavity,” “within the vagina,” and “vaginal canal” refer to the internal genitalia of the human female in the pudendal region of the body. The term “vaginal canal” is not intended to include the interlabial space including the floor of the vestibule. The externally visible genitalia generally are not included within the term “vaginal canal” as used herein.

As used herein, the term “hymen ring” refers to the demarcation between the vagina and the external genitalia and is identified by the position of the hymen or residual tissue of the hymen.

As used herein, the term “proper placement” refers to the vertical position of the tampon inside the vaginal cavity, wherein the base of the tampon (i.e., the main absorbent material) is positioned at least about 5 mm above the hymen ring and more preferably greater than about 10 mm above the hymen ring. The base of the tampon does not include any overwrap, secondary absorbent member, or withdrawal cord which extends beyond the tampon's main absorbent material.

The term “fully expelled” refers to as the base of the tampon disengaged from the all portions of the tampon applicator when the tampon positioning member is at the proximal position. The base of the tampon does not include any overwrap, secondary absorbent member, or withdrawal cord which extends beyond the tampon's main absorbent material.

The term “partially expelled,” as used herein, is meant that the base of the tampon partially retained within a portion of the applicator when the tampon positioning member is at a proximal position. The base of the tampon does not include any overwrap, secondary absorbent member, or withdrawal cord which extends beyond the tampon's main absorbent material.

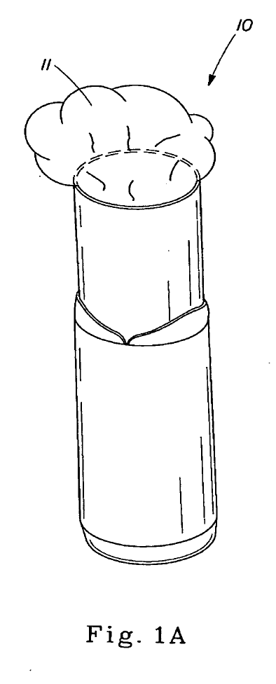

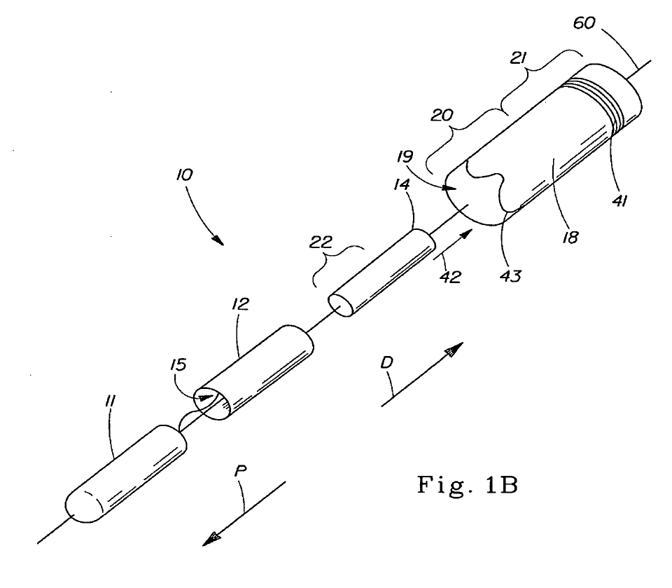

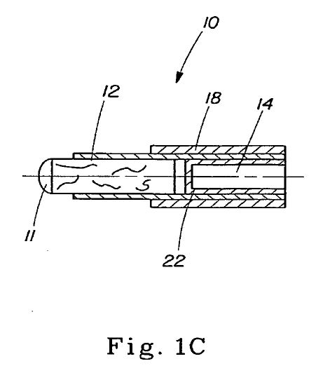

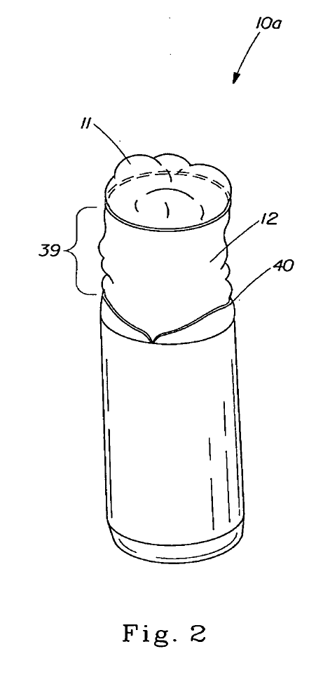

The terms “distal” and “proximal,” respectively designated P and D in FIG. 1b, herein respectively refer to directions away from and towards the body of the tampon wearer unless otherwise specified.

The term “joined” or “attached” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

B. Tampon Applicator of the Present Invention

Referring to FIG. 1a, a tampon applicator 10 is shown which is designed to position tampon 11 to achieve proper placement in the vaginal cavity.

FIG. 1b is an exploded view of FIG. 1a of the tampon applicator 10 along a longitudinal axis 60. The tampon applicator 10 is designed to provide a comfortable means of inserting a tampon 11 into a human's vagina. The tampon applicator 10 can be used with any type of tampon 11. For example, the tampon 11 could be a self-sustaining tampon or a deformable, fluid-permeable bag tampon. Generally, the tampon applicator 10 includes a tampon 11, a tampon housing member 12, a cover member 18, and a tampon positioning member 14.

A hollow interior 15 of the tampon housing member 12 can contain at least a portion of the tampon 11. The tampon housing member 12 can be telescopically slideable within a hollow interior 19 of the cover member 18.

The cover member 18 covers at least a portion of the tampon housing member 12 and can be used to handle or grip the tampon applicator 10 during insertion into the vaginal cavity. The cover member 18 has a top end 20 and a bottom end 21 opposed to the top end 20. The top end 20 is the most proximal end of the cover member 18 along the longitudinal axis 60. The cover member 18 can have a guard 43 located at the top end 20 of the cover member 18 that pushes or “plows” tissue of a wearer away from a lapping position 40 (seen in FIG. 2). The lapping position 40 (shown in FIG. 2), which is located between the tampon housing member 12 and the top end 20 of the cover member 18, minimizes the risk of pinching the body tissue of the tampon wearer.

The tampon positioning member 14 positions the tampon 11 along the length of the tampon housing member 12 so that during expulsion of the tampon 11, the tampon 11 will travel a pre-determined distance. The tampon positioning member 14 can be disposed within the hollow interior 15 of the tampon housing member 12. The tampon positioning member 14 has a first end 22 and a second end 23 opposed to the first end 22. The first end 22 of the tampon positioning member 14 can contact the tampon 11 during expulsion of the tampon 11 from the tampon housing member 12. The second end 23 of the tampon positioning member 14 is the most distal end along the longitudinal axis 60.

To use the tampon applicator of the present invention, the user can insert the tampon applicator into the vaginal cavity by holding a gripping area 41 located in the end 21 of the cover member 18 with the wearer's thumb and index finger. Typically, a perimeter 42 of the grip region 41 can be of any shape. During insertion, as the tampon housing member 12 slides within the hollow interior 19 of the cover member 18, the tampon 11 becomes more exposed within the vagina. Typically, the guard 43, as noted above, is placed on the top end 20 of the cover member 18 to push or “plow” the body tissue away from the lapping position 40 (shown in FIG. 2).

FIG. 1c shows a cross-sectional view of the tampon applicator 10. Specifically, the tampon 11, the tampon housing member 12, the cover member 18, and the first end 22 of the tampon positioning member 14 are shown.

FIG. 2 shows an alternative embodiment of the tampon applicator of the present invention. The tampon housing member 12 of tampon applicator 10A comprises an accordion collapse zone 39 to house the tampon 11.

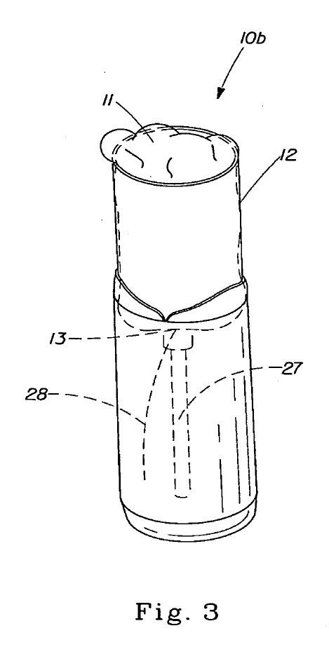

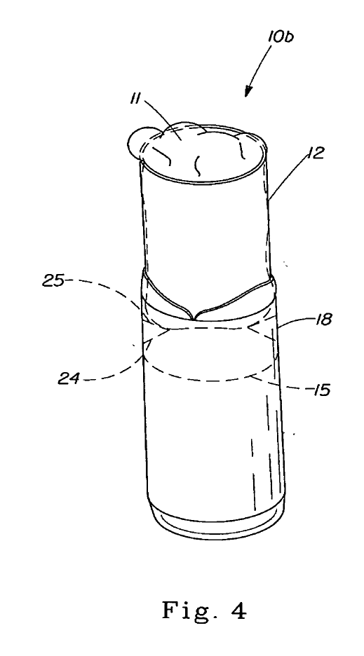

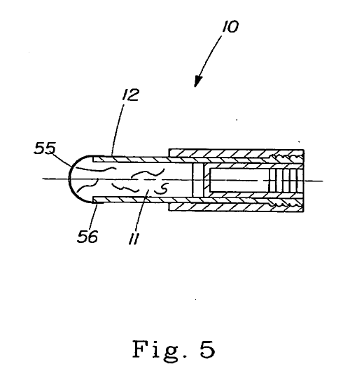

FIG. 3 shows an alternative embodiment of the tampon applicator of the present invention. Tampon applicator 10B comprises a tampon positioning member in the form of a rod 27. The rod 27 positions the tampon 11 along the length of the tampon applicator 10B. The tampon 11 can rest on top of the rod 27. Specifically, a base 13 of the tampon 11 can rest on top of the rod 27. The base 13 of the tampon 11 is located adjacent a string 28. The string 28 is attached to the tampon 11. The string 28 can extend back through the tampon housing member 12. FIG. 4 shows another alternative embodiment of the tampon applicator of the present invention. Tampon applicator 10C comprises a tampon positioning member in the form of projections 24. The projections 24 can extend inward from the hollow interior 15 of the cover member 18. The tampon 11 can rest on a first end 25 of the projections 24. Alternatively, the projections 24 can be monolithically formed on the cover member 18.

In addition to delivering menstrual tampons into the vaginal cavity, it should be noted that the tampon applicator of the present invention can be used to deliver any other type of absorbent or nonabsorbent object to any suitable cavity. For example, the tampon applicator of the present invention could be used to insert incontinence inserts. An “incontinence insert,” as used herein refers to devices specifically designed, configured, and/or adapted for placement into a vagina in order to reduce the occurrence and/or severity of female urinary incontinence. While incontinence inserts are typically made of non-absorbent materials, at least partially absorbent materials may also be used. However, because there is no intent to absorb bodily fluids, and because the incontinence inserts are adapted and configured to provide structural support to the musculature and body tissues located near the urethra-vaginal myfascial area, incontinence inserts are readily distinguishable from catamenial tampons.

Below will detail each component of the tampon applicator of the present invention.

i. Tampon Housing Member

Referring to FIG. 1b, the tampon housing member 12 houses or contains the tampon 11. The tampon housing member 12 has the hollow interior 15. The manufacturer of the tampon applicator 10 can vary the configuration of the tampon housing member 12 and the length of the tampon housing member 12. The configuration of the tampon housing member 12 is preferably created to house or contain the tampon 11. Furthermore, the shape or configuration of the tampon housing member 12 can vary as long as a portion of the tampon 11 is partially contained by the tampon housing member 12. One skilled in the art can imagine other configurations of the tampon housing member 12, for example, cylindrical or curved like a banana or any other suitable shape possible as long as any such shape would work effectively to house or partially contain the tampon 11, properly aiding in the expulsion of the tampon 11 from the tampon applicator 10, and comfortably inserting the tampon 11 into the vaginal cavity. Accordingly, the tampon housing member 12 can be of any suitable cross-sectional shape including, but not limited to, circular, oval, flattened circular, elliptical, and any combination thereof. The diameter of the tampon housing member 12 can be varied to accommodate different absorbency tampons. For example, higher absorbency tampons can have larger diameters resulting in the tampon housing member 12 having a larger diameter to house the higher absorbent tampon. In addition, the tampon housing member 12 can have varying diameters to conform to the profiled shape of the enclosed tampon 11.

The length of the tampon housing member 12 can also vary. The tampon housing member 12 should be of a sufficient length to house or contain at least a portion of the tampon 11 prior to insertion of the tampon applicator 10 into the vaginal cavity. The length of the tampon housing member 12 can be between from about 20 mm to about 89 mm. In one non-limiting example, the length of the tampon housing member 12 is about 30 mm. In yet another non-limiting example, the length of the tampon housing member 12 is about 65 mm.

As shown in FIG. 2, in another non-limiting example, the tampon housing member 12 can comprise the accordion collapse zone 39. This accordion collapse zone 39, during insertion, can collapse with the force of insertion by the user further exposing the tampon 11 to vaginal tissues. The accordion collapse zone 39 can be made from any suitable type of material known in the art which will bend or fold when the insertion force is applied. For example, the accordion collapse zone 39 can be made from a polymer film or paper.

ii Cover Member

Referring to FIG. 1b, the cover member 18 is external to the tampon housing member 12. The cover member 18 has the hollow interior 19, the top end 20, and the bottom end 21 opposed to the top end 20. The top end 20 is the most proximal end of the cover member 18 along the longitudinal axis 60.

At least a portion of the hollow interior 19 of the cover member 18 can engage with at least a portion of the exterior of the tampon housing member 12. In one embodiment, the cover member 18 can be slideable with at least a portion of the tampon housing member 12.

Alternatively, the cover member 18 can be fixedly joined to the tampon positioning member 14 in any way known in the art. Also, alternatively, the cover member 18 can be monolithic with the tampon positioning member 14, for example, by molding the cover member 18 and the tampon positioning member 14 as one piece.

The manufacturer of tampon applicator 10 can vary the configuration of the cover member 18 and the length of the cover member 18.

It is further noted herein that the shape or configuration of the cover member 18 can vary as long as a portion of the tampon housing member 12 can be slideable within the cover member 18. One skilled in the art can imagine that the cover member 18 might be cylindrical or curved like a banana or any other suitable shape can be possible as long as any such shape would work effectively in expelling the tampon 11 from the tampon applicator 10. The cover member 18 can be of any suitable cross-sectional shape. Suitable cross-sectional shapes can include, but are not limited to, circular, oval, flattened circular, elliptical, and any combination thereof.

Also, the cover member 18 can have many designs. As shown in FIG. 1b, in one embodiment, the cover member 18 can be scalloped at the top end 20. The top end 20 is preferably designed to minimize the risk of pinching the vaginal tissue of the wearer. In an alternative embodiment, the top end 20 of the cover member 18 can have a feathered thickness edge.

The cover member 18 can be of any length. In one non-limiting example, the length of the cover member 18 is about 30 mm. In yet another non-limiting example, the length of the cover member 18 is about 65 mm.

Referring again to FIG. 1b, the cover member 18 can comprise the grip region 41 having a perimeter 42. The perimeter 42 of the grip region 41 can take essentially any desired shape, including oval, circular, and various other suitable geometric patterns. The gripping region 41 can be created, for example, by outward projections, raised surfaces, or by impressing or compressing the surfaces. The grip region 41 can be substantially circularly shaped, but can also take on more angular formations such as squared. The surface of the grip region 41 can be any kind of surface known in the art. This surface area can provide a desired frictional resistance during insertion of the tampon applicator 10 into the vaginal cavity. Moreover, this surface area can have, for example, a dove pattern created via surface texturing, impressions, and/or indentations.

Furthermore, it is preferable that the grip region 41 be of sufficient dimension to substantially accept a user's grip. As used in the specification and claims, the term “user's grip” means any way of holding the tampon applicator 10 in a hand, e.g., between a thumb and a finger. The grip region 41 need not necessarily extend completely around the perimeter of the outer member 23. For example, a space can be provided for a decorative marking or a Trademark character. As shown in FIG. 1b, the grip region 41 provides for secure handling of the cover member 18.

The cover member 18 can be included in the embodiment of the tampon applicator for several reasons. First, the cover member 18 can be used to handle or grip the tampon applicator during insertion into the vaginal cavity. Second, the cover member 18 can provide the gripping area 41 to the tampon applicator which allows a larger portion of the tampon applicator to be inserted into the vaginal cavity because the cover member 18 allows the tampon 11 to travel a greater distance. The gripping area 41, generally, is not inserted into the vaginal cavity and creates additional length to the tampon applicator. Thus, the cover member 18 can provide additional travel distance by the tampon applicator. Third, the cover member 18 provides more stability during the insertion of the tampon applicator. Finally, referring to FIG. 1b, the guard 43 located at the top end 20 of the cover member 18 minimizes the risk of pinching the tissue of the wearer. As discussed above, the guard 43 pushes or plows” tissue away from the lapping position 40 (seen in FIG. 2). The lapping position 40 (seen in FIG. 2) is located between the tampon housing member 12 and the top end 20 of the cover member 18. Thus, if the cover member 18 is not included, the tampon applicator 10 could inadvertently draw body tissue into the lapping position 40 (seen in FIG. 2) and/or pinch the tissue of the wearer during insertion of the tampon applicator 10 into the vaginal cavity.

iii Tampon Positioning Member

Referring to FIG. 1b, the tampon positioning member 14 positions the tampon 11 along the length of the tampon housing member 12 so that during expulsion the tampon 11 will travel a pre-determined distance. This predetermined distance ultimately results in the tampon 11 being properly positioned in the vagina. Moreover, this position allows sufficient expulsion such that when the tampon 11 is expelled at least a portion of the tampon 11 is retained within the vagina by body forces.

The tampon positioning member 14 has a first end 22 and a second end 23 opposed to the first end 22. The first end 22 is the most proximal end of the tampon positioning member 14 along the longitudinal axis 60. In addition, the first end 22 of the tampon positioning member 14 can contact the tampon 11 during expulsion of the tampon 11 from the tampon housing member 12. The second end 23 of the tampon positioning member 14 is the most distal end along the longitudinal axis 60.

Referring to FIG. 1c, during expulsion, the tampon 11 is in contact with the tampon positioning member 14. In one non-limiting example, at least a portion of the tampon 11 can rest against the first end 22 of the tampon positioning member 14 during expulsion of the tampon 11 from the tampon applicator 10. However, the tampon 11 may or may not be in contact with the tampon positioning member 14 before expulsion of the tampon 11 from the tampon applicator 10. Referring to FIG. 1b, for example, in one non-limiting example, before expulsion of the tampon 11 from the tampon applicator 10, the tampon positioning member 14 can be located in the hollow interior 15 of the cover member 18 without any contact with the tampon 11.

The tampon positioning member 14 can be fixedly joined to the tampon housing member 12 and/or the cover member 18 in any way known in the art. The tampon positioning member 14 can also be monolithic with the tampon housing member 12, for example, by molding the tampon positioning member 14 and the tampon housing member 12 as one piece. Alternatively, the tampon positioning member 14 can be monolithic with the cover member 18, for example, by molding the tampon positioning member 14 and the cover member 18 as one piece. In one non-limiting example, the tampon positioning member 14 can be fixedly joined to the cover member 18 and/or tampon housing member 12 by friction fitting that snaps parts together, gluing, and/or melting. In another non-limiting example, the tampon positioning member 14 can also be a continuation of the cover member 18 by folding or compressing a portion of the cover member 18 and turning the cover member 18 inwards upon itself to create a tube inside a tube. Likewise, the tampon positioning member 14 can also be a continuation of the tampon housing member 12 by folding or compressing a portion of the tampon housing member 12 and turning the tampon housing member 12 inwards upon itself to create a tube inside a tube.

It is further noted herein that the shape or configuration of the tampon positioning member 14 can vary as long as the tampon positioning member 14 aids in positioning the tampon 11 along the length of the tampon housing member 12 to provide proper placement of the tampon 11 within a vaginal cavity. As shown in FIG. 1b, one embodiment of the tampon positioning member 14 includes an elongated tampon positioning member 14 of a generally tubular shape. Also, as shown in FIG. 3, the tampon positioning member can be a rod 27. The base 13 of the tampon 11 can rest on top of the rod 27. The base 13 of the tampon 11 is located adjacent the string 28. The string 28 is attached to the tampon 11. The string 28 can extend back through the tampon housing member 12. In another embodiment, the tampon positioning member 14 can be slideable within the tampon housing member 12.

As shown in FIG. 4, in another alternative embodiment, the tampon positioning member can also comprise projections 24 extending inward from the hollow interior 15 of the cover member 18. The projections 24 can be of any shape or size as long as the projections 24 allow the tampon 11 to rest on the projections 24. Specifically, the tampon 11 can rest on a first end 25 of the projections 24. The projections 24 can be fixedly joined to the cover member 18 and/or tampon housing member 12 in any way known in the art.

It is further noted herein that the length of the tampon positioning member 14 can vary as long as the tampon positioning member 14 aids the tampon 11 in being located in the pre-determined position within the tampon housing member 12 so that proper placement within the vaginal cavity is achieved. In one non-limiting example, the tampon positioning member 14 can be from about 36 millimeters to about 63 millimeters.

C. Tampon Applicator Materials

Different tampon applicator parts can be constructed from different materials and processes. The tampon applicator or any part of the tampon applicator can be formed of a spirally wound, convolutedly wound, or longitudinally seamed hollow tube that is formed from paper, paperboard, cardboard, or any combination thereof. The tampon applicator or any part of the tampon applicator can also be injection molded, extruded, or formed from flexible plastic, such as thermoformed from plastic sheet or folded or wound from plastic film.

The tampon applicator or any part of the tampon applicator can be constructed from a single ply of material or can be formed from two or more plies that are bonded together to form a laminate. The use of two or more plies or layers is preferred for it enables the manufacturer to use certain materials in the various layers that can enhance the performance of the tampon applicator or any part of the tampon applicator. When two or more plies are utilized, all the plies can be spirally wound, convolutedly wound, or longitudinally seamed to form an elongated cylinder. The tampon applicator or any part of the tampon applicator can be constructed using a smooth thin ply of material on the outside or exterior surface that surrounds a coarser and possibly thicker ply. When the tampon applicator or any part of the tampon applicator contains at least three plies, the middle ply can be the thicker ply, and the interior and exterior plies can be smooth and/or slippery to facilitate expulsion of the tampon and to facilitate insertion of the tampon applicator or any part of the tampon applicator into a woman's vagina. The tampon applicator or any part of the tampon applicator should contain one to four plies, although more plies can be utilized if desired.

The plies forming the tampon applicator or any part of the tampon applicator can be held together by an adhesive, such as glue, heat, pressure, ultrasonic, etc. The adhesive can be either water-soluble or water-insoluble. A water-soluble adhesive is preferred for environmental reasons in that the tampon applicator or any part of the tampon applicator will quickly break apart when it is immersed in water. Such immersion will occur should the tampon applicator or any part of the tampon applicator be disposed of by flushing it down a toilet. Exposure of the tampon applicator or any part of the tampon applicator to a municipal waste treatment plant wherein soaking in water, interaction with chemicals, and agitation all occur, will cause the tampon applicator or any part of the tampon applicator to break apart and evenly disperse in a relatively short period of time.

### EXAMPLES

The following is a listing of examples illustrating various embodiments of the present invention. It would be obvious to those skilled in the art that various other changes and modifications can be made without departing from the spirit and scope of the invention.

### Example 1

The tampon applicator of the present invention can comprise the tampon housing member of the length of 56 mm, the cover member of the length of 55 mm, a tampon positioning member of the length of 45 mm, and a tampon of the length of 44 mm.

### Example 2

The tampon applicator of the present invention can comprise the tampon housing member of the length of 50 mm, the cover member of the length of 59 mm, a tampon positioning member of the length of 45 mm, and a tampon of the length of 46 mm.

### Example 3

The tampon applicator of the present invention can comprise the tampon housing member of the length of 50 mm, the cover member of the length of 64 mm, a tampon positioning member of the length of 53.6 mm, and a tampon of the length of 45 mm.

### Example 4

The tampon applicator of the present invention can comprise the tampon housing member of the length of 42 mm, the cover member of the length of 55 mm, a tampon positioning member of the length of 45 mm, and a tampon of the length of 30 mm.

### Example 5

The tampon applicator of the present invention can comprise the tampon housing member of the length of 60 mm, the cover member of the length of 59 mm, a tampon positioning member of the length of 45 mm, and a tampon of the length of 53 mm.

### Example 6

The tampon applicator of the present invention can comprise the tampon housing member of the length of 58 mm, the cover member of the length of 69 mm, a tampon positioning member of the length of 57 mm, and a tampon of the length of 53 mm.

### Example 7

The tampon applicator of the present invention can comprise the tampon housing member of the length of 45 mm, the cover member of the length of 67 mm, a tampon positioning member of the length of 57 mm, and a tampon of the length of 30 mm.

### Example 8

The tampon applicator of the present invention can comprise the tampon housing member of the length of 50 mm, the cover member of the length of 60 mm, a tampon positioning member of the length of 50 mm, and a tampon of the length of 40 mm.

### Example 9

The tampon applicator of the present invention can comprise the tampon housing member of the length of 45 mm, the cover member of the length of 55 mm, a tampon positioning member of the length of 45 mm, and a tampon of the length of 33 mm.

### Example 10

The tampon applicator of the present invention can comprise the tampon housing member of the length of 45 mm, the cover member of the length of 60 mm, a tampon positioning member of the length of 50 mm, and a tampon of the length of 35 mm.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon applicator for positioning a tampon inside a vaginal cavity, said applicator comprising:
a cover member for covering at least a portion of a tampon housing member; said tampon housing member is telescopically mounted within said cover member;
said tampon being partially housed within a tampon housing member, said tampon extending beyond said tampon housing member; and
said tampon positioning member for positioning said tampon within said housing member, said tampon positioning member being disposed inside said housing member and co-axially with said tampon, wherein said tampon positioning member being fixedly joined to said cover member.

2. The tampon applicator of claim 1 wherein said housing member is slideable over said positioning member.

3. The tampon applicator of claim 1 wherein said cover member comprises a bottom end positioned adjacent said housing member.

4. The tampon applicator of claim 1 wherein said housing member is slidably joined to said cover member.

5. The tampon applicator of claim 1 wherein said housing member is fixedly joined to said cover member.

6. The tampon applicator of claim 1 wherein a collar is joined to said cover member

7. A tampon applicator for positioning a tampon inside a vaginal cavity, said applicator comprising:
a cover member for covering at least a portion of a tampon housing member, wherein said tampon housing member is telescopically mounted within a cover member;
said tampon being partially housed within said tampon housing member, said tampon extending beyond said tampon housing member; and
said tampon positioning member for positioning said tampon within said housing member, said tampon positioning member being disposed inside said housing member and co-axially with said tampon, and wherein said tampon positioning member is fixedly joined to said tampon housing member.

8. The tampon applicator of claim 6 wherein said housing member is slideable over said positioning member.

9. The tampon applicator of claim 6 wherein said cover member comprises a bottom end positioned adjacent said housing member.

10. The tampon applicator of claim 6 wherein said housing member is joined to said cover member.

11. The tampon applicator of claim 9 wherein said housing member is fixedly joined to said cover member.

12. The tampon applicator of claim 9 wherein a collar is joined to said cover member.

13. A tampon applicator for positioning a tampon inside a vaginal cavity, said applicator comprising:
a cover member for covering at least a portion of said tampon housing member; said tampon housing member is telescopically mounted within said cover member;
a tampon being partially housed within said tampon housing member, said tampon extending beyond said tampon housing member;
a tampon positioning member for positioning said tampon within said housing member, said tampon positioning member being disposed inside said housing member and co-axially with said tampon, said tampon positioning member being fixedly joined to said tampon housing member
wherein said tampon housing member slides in a distal direction at a force of ?N upon insertion.
